SEQUENCE LISTING (28 sequences)

SEQ ID NO: 1 - DNA, 3160 bp, Artificial Sequence
  RSF1010 ori
atgaagaacgacaggactttgcaggccataggccgacagctcaaggccatgggctgtgagcgcttcgatatcggcgtcagggacgcacccaccggccagatgatgaaccgggaatggtcagccgccgaagtgctccagaacacgccatggctcaagcggatgaatgcccagggcaatgacgtgtatatcaggcccgccgagcaggagcggcatggtctggtgctggtggacgacctcagcgagtttgacctggatgacatgaaagccgagggccgggagcctgccctggtagtggaaaccagcccgaagaactatcaggcatgggtcaaggtggccgacgccgcaggcggtgaacttcgggggcagattgcccggacgctggccagcgagtacgacgccgacccggccagcgccgacagccgccactatggccgcttggcgggcttcaccaaccgcaaggacaagcacaccacccgcgccggttatcagccgtgggtgctgctgcgtgaatccaagggcaagaccgccaccgctggcccggcgctggtgcagcaggctggccagcagatcgagcaggcccagcggcagcaggagaaggcccgcaggctggccagcctcgaactgcccgagcggcagcttagccgccaccggcgcacggcgctggacgagtaccgcagcgagatggccgggctggtcaagcgcttcggtcatgacctcagcaagtgcgactttatcgccgcgcagaagctggccagccggggccgcagtgccgaggaaatcggcaaggccatggccgaggccagcccagcgctggcagagcgcaagcccggccacgaagcggattacatcgagcgcaccgtcagcaaggtcatgggtctgcccagcgtccagcttgcgcgggccgagctggcacgggcaccggcaccccgccagcgaggcatggacaggggcgggccagatttcagcatgtagtgcttgcgttggtactcacgcctgttatactatgagtactcacgcacagaagggggttttatggaatacgaaaaaagcgcttcagggtcggtctacctgatcaaaagtgacaagggctattggttgcccggtggctttggttatacgtcaaacaaggccgaggctggccgcttttcagtcgctgatatggccagccttaaccttgacggctgcaccttgtccttgttccgcgaagacaagcctttcggccccggcaagtttctcggtgactgatatgaaagaccaaaaggacaagcagaccggcgacctgctggccagccctgacgctgtacgccaagcgcgatatgccgagcgcatgaaggccaaagggatgcgtcagcgcaagttctggctgaccgacgacgaatacgaggcgctgcgcgagtgcctggaagaactcagagcggcgcagggcgggggtagtgaccccgccagcgcctaaccaccaactgcctgcaaaggaggcaatcaatggctacccataagcctatcaatattctggaggcgttcgcagcagcgccgccaccgctggactacgttttgcccaacatggtggccggtacggtcggggcgctggtgtcgcccggtggtgccggtaaatccatgctggccctgcaactggccgcacagattgcaggcgggccggatctgctggaggtgggcgaactgcccaccggcccggtgatctacctgcccgccgaagacccgcccaccgccattcatcaccgcctgcacgcccttggggcgcacctcagcgccgaggaacggcaagccgtggctgacggcctgctgatccagccgctgatcggcagcctgcccaacatcatggccccggagtggttcgacggcctcaagcgcgccgccgagggccgccgcctgatggtgctggacacgctgcgccggttccacatcgaggaagaaaacgccagcggccccatggcccaggtcatcggtcgcatggaggccatcgccgccgataccgggtgctctatcgtgttcctgcaccatgccagcaagggcgcggccatgatgggcgcaggcgaccagcagcaggccagccggggcagctcggtactggtcgataacatccgctggcagtcctacctgtcgagcatgaccagcgccgaggccgaggaatggggtgtggacgacgaccagcgccggttcttcgtccgcttcggtgtgagcaaggccaactatggcgcaccgttcgctgatcggtggttcaggcggcatgacggcggggtgctcaagcccgccgtgctggagaggcagcgcaagagcaagggggtgccccgtggtgaagcctaagaacaagcacagcctcagccacgtccggcacgacccggcgcactgtctggcccccggcctgttccgtgccctcaagcggggcgagcgcaagcgcagcaagctggacgtgacgtatgactacggcgacggcaagcggatcgagttcagcggcccggagccgctgggcgctgatgatctgcgcatcctgcaagggctggtggccatggctgggcctaatggcctagtgcttggcccggaacccaagaccgaaggcggacggcagctccggctgttcctggaacccaagtgggaggccgtcaccgctgaatgccatgtggtcaaaggtagctatcgggcgctggcaaaggaaatcggggcagaggtcgatagtggtggggcgctcaagcacatacaggactgcatcgagcgcctttggaaggtatccatcatcgcccagaatggccgcaagcggcaggggtttcggctgctgtcggagtacgccagcgacgaggcggacgggcgcctgtacgtggccctgaaccccttgatcgcgcaggccgtcatgggtggcggccagcatgtgcgcatcagcatggacgaggtgcgggcgctggacagcgaaaccgcccgcctgctgcaccagcggctgtgtggctggatcgaccccggcaaaaccggcaaggcttccatagataccttgtgcggctatgtctggccgtcagaggccagtggttcgaccatgcgcaagcgccgcaagcgggtgcgcgaggcgttgccggagctggtcgcgctgggctggacggtaaccgagttcgcggcgggcaagtacgacatcacccggcccaaggcggcaggctga

SEQ ID NO: 2 - DNA, 534 bp, Artificial Sequence
  GmR
ttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacat

SEQ ID NO: 3 - DNA, 879 bp, Artificial Sequence
  araC gene
atggctgaagcgcaaaatgatcccctgctgccgggatactcgtttaatgcccatctggtggcgggtttaacgccgattgaggccaacggttatctcgatttttttatcgaccgaccgctgggaatgaaaggttatattctcaatctcaccattcgcggtcagggggtggtgaaaaatcagggacgagaatttgtttgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacggggttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcttgtcgccgtcgcgtctgtcacatcttttccgccagcagttagggattagcgtcttaagctggcgcgaggaccaacgtatcagccaggcgaagctgcttttgagcaccacccggatgcctatcgccaccgtcggtcgcaatgttggttttgacgatcaactctatttctcgcgggtatttaaaaaatgcaccggggccagcccgagcgagttccgtgccggttgtgaagaaaaagtgaatgatgtagccgtcaagttgtcataa

SEQ ID NO: 4 - DNA, 28 bp, Artificial Sequence
  araBAD promoter
gacgctttttatcgcaactctctactgt

SEQ ID NO: 5 - DNA, 417 bp, Artificial Sequence
  Gam gene
atggatattaatactgaaactgagatcaagcaaaagcattcactaaccccctttcctgttttcctaatcagcccggcatttcgcgggcgatattttcacagctatttcaggagttcagccatgaacgcttattacattcaggatcgtcttgaggctcagagctgggcgcgtcactaccagcagctcgcccgtgaagagaaagaggcagaactggcagacgacatggaaaaaggcctgccccagcacctgtttgaatcgctatgcatcgatcatttgcaacgccacggggccagcaaaaaatccattacccgtgcgtttgatgacgatgttgagtttcaggagcgcatggcagaacacatccggtacatggttgaaaccattgctcaccaccaggttgatattgattcagaggtataa

SEQ ID NO: 6 - DNA, 786 bp, Artificial Sequence
  Bet gene
atgagtactgcactcgcaacgctggctgggaagctggctgaacgtgtcggcatggattctgtcgacccacaggaactgatcaccactcttcgccagacggcatttaaaggtgatgccagcgatgcgcagttcatcgcattactgatcgttgccaaccagtacggccttaatccgtggacgaaagaaatttacgcctttcctgataagcagaatggcatcgttccggtggtgggcgttgatggctggtcccgcatcatcaatgaaaaccagcagtttgatggcatggactttgagcaggacaatgaatcctgtacatgccggatttaccgcaaggaccgtaatcatccgatctgcgttaccgaatggatggatgaatgccgccgcgaaccattcaaaactcgcgaaggcagagaaatcacggggccgtggcagtcgcatcccaaacggatgttacgtcataaagccatgattcagtgtgcccgtctggccttcggatttgctggtatctatgacaaggatgaagccgagcgcattgtcgaaaatactgcatacactgcagaacgtcagccggaacgcgacatcactccggttaacgatgaaaccatgcaggagattaacactctgctgatcgccctggataaaacatgggatgacgacttattgccgctctgttcccagatatttcgccgcgacattcgtgcatcgtcagaactgacacaggccgaagcagtaaaagctcttggattcctgaaacagaaagccgcagagcagaaggtggcagcatga

SEQ ID NO: 7 - DNA, 681 bp, Artificial Sequence
  Exo gene
atgacaccggacattatcctgcagcgtaccgggatcgatgtgagagctgtcgaacagggggatgatgcgtggcacaaattacggctcggcgtcatcaccgcttcagaagttcacaacgtgatagcaaaaccccgctccggaaagaagtggcctgacatgaaaatgtcctacttccacaccctgcttgctgaggtttgcaccggtgtggctccggaagttaacgctaaagcactggcctggggaaaacagtacgagaacgacgccagaaccctgtttgaattcacttccggcgtgaatgttactgaatccccgatcatctatcgcgacgaaagtatgcgtaccgcctgctctcccgatggtttatgcagtgacggcaacggccttgaactgaaatgcccgtttacctcccgggatttcatgaagttccggctcggtggtttcgaggccataaagtcagcttacatggcccaggtgcagtacagcatgtgggtgacgcgaaaaaatgcctggtactttgccaactatgacccgcgtatgaagcgtgaaggcctgcattatgtcgtgattgagcgggatgaaaagtacatggcgagttttgacgagatcgtgccggagttcatcgaaaaaatggacgaggcactggctgaaattggttttgtatttggggagcaatggcgatga

SEQ ID NO: 8 - DNA, 73 bp, Artificial Sequence
  Tet promoter
taattcctaatttttgttgacactctatcgttgatagagttattttaccactccctatcagtgatagagaaaa

SEQ ID NO: 9 - DNA, 4107 bp, Artificial Sequence
  SpCas9 gene
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 10 - DNA, 3156 bp, Artificial Sequence
  SaCas9 gene
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaag

SEQ ID NO: 11 - DNA, 3903 bp, Artificial Sequence
  FnCas12a gene
atgtcaatttatcaagaatttgttaataaatatagtttaagtaaaactctaagatttgagttaatcccacagggtaaaacacttgaaaacataaaagcaagaggtttgattttagatgatgagaaaagagctaaagactacaaaaaggctaaacaaataattgataaatatcatcagttttttatagaggagatattaagttcggtttgtattagcgaagatttattacaaaactattctgatgtttattttaaacttaaaaagagtgatgatgataatctacaaaaagattttaaaagtgcaaaagatacgataaagaaacaaatatctgaatatataaaggactcagagaaatttaagaatttgtttaatcaaaaccttatcgatgctaaaaaagggcaagagtcagatttaattctatggctaaagcaatctaaggataatggtatagaactatttaaagccaatagtgatatcacagatatagatgaggcgttagaaataatcaaatcttttaaaggttggacaacttattttaagggttttcatgaaaatagaaaaaatgtttatagtagcaatgatattcctacatctattatttataggatagtagatgataatttgcctaaatttctagaaaataaagctaagtatgagagtttaaaagacaaagctccagaagctataaactatgaacaaattaaaaaagatttggcagaagagctaacctttgatattgactacaaaacatctgaagttaatcaaagagttttttcacttgatgaagtttttgagatagcaaactttaataattatctaaatcaaagtggtattactaaatttaatactattattggtggtaaatttgtaaatggtgaaaatacaaagagaaaaggtataaatgaatatataaatctatactcacagcaaataaatgataaaacactcaaaaaatataaaatgagtgttttatttaagcaaattttaagtgatacagaatctaaatcttttgtaattgataagttagaagatgatagtgatgtagttacaacgatgcaaagtttttatgagcaaatagcagcttttaaaacagtagaagaaaaatctattaaagaaacactatctttattatttgatgatttaaaagctcaaaaacttgatttgagtaaaatttattttaaaaatgataaatctcttactgatctatcacaacaagtttttgatgattatagtgttattggtacagcggtactagaatatataactcaacaaatagcacctaaaaatcttgataaccctagtaagaaagagcaagaattaatagccaaaaaaactgaaaaagcaaaatacttatctctagaaactataaagcttgccttagaagaatttaataagcatagagatatagataaacagtgtaggtttgaagaaatacttgcaaactttgcggctattccgatgatatttgatgaaatagctcaaaacaaagacaatttggcacagatatctatcaaatatcaaaatcaaggtaaaaaagacctacttcaagctagtgcggaagatgatgttaaagctatcaaggatcttttagatcaaactaataatctcttacataaactaaaaatatttcatattagtcagtcagaagataaggcaaatattttagacaaggatgagcatttttatctagtatttgaggagtgctactttgagctagcgaatatagtgcctctttataacaaaattagaaactatataactcaaaagccatatagtgatgagaaatttaagctcaattttgagaactcgactttggctaatggttgggataaaaataaagagcctgacaatacggcaattttatttatcaaagatgataaatattatctgggtgtgatgaataagaaaaataacaaaatatttgatgataaagctatcaaagaaaataaaggcgagggttataaaaaaattgtttataaacttttacctggcgcaaataaaatgttacctaaggttttcttttctgctaaatctataaaattttataatcctagtgaagatatacttagaataagaaatcattccacacatacaaaaaatggtagtcctcaaaaaggatatgaaaaatttgagtttaatattgaagattgccgaaaatttatagatttttataaacagtctataagtaagcatccggagtggaaagattttggatttagattttctgatactcaaagatataattctatagatgaattttatagagaagttgaaaatcaaggctacaaactaacttttgaaaatatatcagagagctatattgatagcgtagttaatcagggtaaattgtacctattccaaatctataataaagatttttcagcttatagcaaagggcgaccaaatctacatactttatattggaaagcgctgtttgatgagagaaatcttcaagatgtggtttataagctaaatggtgaggcagagcttttttatcgtaaacaatcaatacctaaaaaaatcactcacccagctaaagaggcaatagctaataaaaacaaagataatcctaaaaaagagagtgtttttgaatatgatttaatcaaagataaacgctttactgaagataagtttttctttcactgtcctattacaatcaattttaaatctagtggagctaataagtttaatgatgaaatcaatttattgctaaaagaaaaagcaaatgatgttcatatattaagtatagatagaggtgaaagacatttagcttactatactttggtagatggtaaaggcaatatcatcaaacaagatactttcaacatcattggtaatgatagaatgaaaacaaactaccatgataagcttgctgcaatagagaaagatagggattcagctaggaaagactggaaaaagataaataacatcaaagagatgaaagagggctatctatctcaggtagttcatgaaatagctaagctagttatagagtataatgctattgtggtttttgaggatttaaattttggatttaaaagagggcgtttcaaggtagagaagcaggtctatcaaaagttagaaaaaatgctaattgagaaactaaactatctagttttcaaagataatgagtttgataaaactgggggagtgcttagagcttatcagctaacagcaccttttgagacttttaaaaagatgggtaaacaaacaggtattatctactatgtaccagctggttttacttcaaaaatttgtcctgtaactggttttgtaaatcagttatatcctaagtatgaaagtgtcagcaaatctcaagagttctttagtaagtttgacaagatttgttataaccttgataagggctattttgagtttagttttgattataaaaactttggtgacaaggctgccaaaggcaagtggactatagctagctttgggagtagattgattaactttagaaattcagataaaaatcataattgggatactcgagaagtttatccaactaaagagttggagaaattgctaaaagattattctatcgaatatgggcatggcgaatgtatcaaagcagctatttgcggtgagagcgacaaaaagttttttgctaagctaactagtgtcctaaatactatcttacaaatgcgtaactcaaaaacaggtactgagttagattatctaatttcaccagtagcagatgtaaatggcaatttctttgattcgcgacaggcgccaaaaaatatgcctcaagatgctgatgccaatggtgcttatcatattgggctaaaaggtctgatgctactaggtaggatcaaaaataatcaagagggcaaaaaactcaatttggttatcaaaaatgaagagtattttgagttcgtgcagaataggaataactaa

SEQ ID NO: 12 - DNA, 589 bp, Artificial Sequence
  ColE1 ori
tttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaa

SEQ ID NO: 13 - DNA, 1191 bp, Artificial Sequence
  TcR
atgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctga

SEQ ID NO: 14 - DNA, 214 bp, Artificial Sequence
  HCE promoter
acagattcccaatctcttgttaaataacgaaaaggcatcaatcaaaacggcggcatgtctttctatattccagcaatgttttataggggacatattgatgaagatgggtatcaccttagtaaaaaaagaattgctataagctgctcttttttgttcgtgatatactgataataaattgaattttcacacttctgaaaaaaggagctggaaccaa

SEQ ID NO: 15 - DNA, 1261 bp, Artificial Sequence
  HRL
agaatcaggttcagggcaatagccacaatggcgcacagggcgatgcccttcaggccccagtcatccgggccgtcaccgctgccgatcagcacgccgccgataccgaacaccagtgtcaccgagacgatcaccaggttgcgtgcctcgcccaggtcaatcttgtggcggatcatggtgttcatgcccaccgctgcaatcgagccgaacagcaggcaaagaatgccgcccatcaccggcaccgggatgctctgcagcagcgcgccgaacttgccgatgaacgcaagggtaatggcgaagaccgccgcccaggtcatgatcttcgggttgtagttcttggtcagcatcaccgcgccggtcacttcggcataggtggtgttgggcgggccgccgaacaggccggctgcagtagtcgccaggccgtcgcccagcaacgtccggtgcaggccaggctttttcaggtagtcacggccggtcacgctgcccactgcaatcaccccgccgatatgctcgatcgccggcgccagcgcaaccgggacgatgaacagaatggcctgccagttgaacgcaggggcggtgaaattgggaatttccagccacggtgcggcagcgatcctggcggtgtcgaccacgccaaaggcgaacgacagggcaaagcccaccagcacgccggagatgatgggtaccaggcggaaaatgcccttgccgaatacggcaacgatcaaggttgtcagcagcgctggcatcgagatcagcatggccgtcttgtacggcatcagtgccgtaccgtccccacctttgcccatcgccatgttggcggcgatcggtgccatggccaggccgatggagatgatcaccgggccgatcaccaccggcggcagcatgcggtcgatgaaaccggtgcctttgatttttaccatcagccccatgaaggtgtacacgaagccggcagccattacgccacccatggtctcggcaaggccgaactggcctttggcgaggatgatcggcgtgatgaaggcaaagctcgaggccaggaacaccggtacctgacgaccggtgaccagctggaatagcagcgtgccgatgccggcggtgaacagcgcgacgtttgggtcgaggccggtgatcaggggcatcagcaccagcgcgccgaatgccacgaagagcatctgcgcgcccgaaacgacctggcgccagagcgggtcgttgaagccgtcctgcatggtcaggcgtccttttgcttggtgccgaagatcttgtc

SEQ ID NO: 16 - DNA, 939 bp, Artificial Sequence
  HRR
aaaaaaaccgcgctagattaatctattcagcctgtgctgtcgtctggtcattctggacgttagtccataaatgcttgatctgtgacgagcggatgcgtacctttgcccgcttttccaaaatgccccccggagcgcgccatgtccgccgatctcgagcatatccgtcaagtcatgcatgaagctgactgcctgtacaccgaagccgaagtcgaagcggccatcgccaaagtcggcgagcagatctgcaaggacctgcacgacaagaacccggttgtgttctgcgtgatgaacggtggcctgatcttctccggcaaactgctgacccacctgcagttcccgctggaagcctcgtacctgcatgctacccgctaccgcaaccagaccagcggtggcgagcttttctggaaggccaagccggaagtgtcgttcatcgaccgtgacgtgttgatcgttgacgatatcctcgacgaaggccacaccctcagcgccatcatcgagttttgcaagcatgccggcgcccgctcggtgtacaccgctgtgctgatcgacaaggaccacgaccgcaaggcaagccctgacctcaaggccaactatgtgggcctgccttgtgtcgatcgctatatcttcggttacggcatggactacaaaggttactggcgcaacgctaacggcatcttcgccgtaaaggggctgtaaccgtgcgccagcctgcgttcatcgaccaatcgctgttcgccgggctggccgaaaaggccgctgccaacccgcgcgggcggcagcaccataactttcacgctatggaagagccctgccaccgcatggccgtcgggctgcaaccaagcacctacattccgccgcatcgccatttgagcgccgacaaggctgaaaccttgatagcgctcaaggggcgttttggcctgttgatcttcgatgaacaag

SEQ ID NO: 17 - DNA, 35 bp, Artificial Sequence
  J23119 promoter
ttgacagctagctcagtcctaggtataatactagt

SEQ ID NO: 18 - DNA, 62 bp, Artificial Sequence
  sgRNA::UPP-Sp
ggatggcggatctcacgagtgttttagagctagaaatagcaagttaaaataaggctagtccg

SEQ ID NO: 19 - DNA, 99 bp, Artificial Sequence
  sgRNA::UPP-Sa
ggcggtgtagatcttcacgtgttttagtactctggaaacagaatctactaaaacaaggcaaaatgccgtgtttatctcgtcaacttgttggcgagattt

SEQ ID NO: 20 - DNA, 39 bp, Artificial Sequence
  sgRNA::UPP-Fn
aatttctactgttgtagattccaccacctcgatgccttc

SEQ ID NO: 21 - DNA, 1737 bp, Artificial Sequence
  HmfH gene
atggacaccccgcgcgagcgtttcgactacgtcattgtcggcggcggtagcgccggctgcgtgttggcgaaccgtctgtcgcaggaccccgccatccgcgtggccttgattgaggccggcgtggataccccgcccgatgccgtgcccgcggaaatcctggactcgtacccgatgccgctgttcttcggcgatcgctacatctggccctcgctgcaagcgcgcgccgtggccggcggccgcagcaaggtctatgagcaaggccgcgtcatgggcggtggctcgagcatcaatgtgcaggccgccaaccgcggcctgccgcgcgactacgatgaatgggccgcgagcggcgccagcggctggagctggcaggatgtgttgccctactttcgtcacctggaacgcgacgtggactacggcaacagccccctgcacggtagccacggcccggtgccgatccgccgcattttgccccaggcctggccgccgttctgcacggaattcgcccatgccatgggccgctcgggtttgtcggccctggccgaccagaacgccgagttcggtgacggttggttcccggcggccttcagcaacctggacgacaaacgcgtcagcacggcgatcgcgtacctggatgccgacacccgccgtcgtgccaacctgcgcatctacgccgagaccaccgtgcgtaagctggtcgtgtcgggccgtgaagcgcgtggcgtgatcgccatgcgcgcggatggttcgcgtctggccctggatgcgggtgaagtcatcgtgagcgccggcgccctgcagtcgccggccatcttgatgcgcgccggcatcggtgatgccggcgcgttgcaagccctgggcatcgaggtggtggccgatcgtcctggtgtcggccgtaatttgcaggaccacccggcgctgaccttctgccagttcctggcgccccagtaccgtatgccgttgtcgcgccgccgtgccagcatgaccgccgcccgctttagcagcggcgtgccgggcggcgaggccagcgatatgtacctgtcgagctcgacccgtgccggctggcacgccctgggcaatcgcctgggcttgtttttcttgtggtgcaaccgcccgttcagccgcggtcaggtgagcttggccggcgcgcagccggacgtgccgcccatggtcgagctgaacctgctggacgatgaacgtgacctgcgccgcatggtggccggcgtgcgtaaactggtccagatcgtgggtgcgtcggccttgcaccaacatcctggtgatttcttcccggccaccttctcgccccgtgtgaaagccctgtcgcgcgtgagccgtggcaatgtgctgctgaccgaactgctgggtgccgtcctggacgtgagcggtccgttgcgccgtagcctgatcgcccgtttcgtgaccggcggcgccaacttggccagcctgctgacggacgaaagcgccctggagggcttcgtgcgtcagtcggtgttcggcgtgtggcatgcctcgggcacctgtcgcatgggtgcccacgccgatcgcagcgccgtcaccgacgccgccggccgcgtccacgatgtgggccgcctgcgtgtgatcgacgccagcctgatgccccgcctgccgaccgccaacaccaacatcccgaccatcatgctggccgagaagatcgcggacaccatgcaggccgagcgccgcgccgtgcgcccggccagcagcgaagtcgcccatcccagc

SEQ ID NO: 22 - DNA, 34 bp, Artificial Sequence
  Flp/Frt sequence
gaagttcctattctctagaaagtataggaacttc

SEQ ID NO: 23 - DNA, 20 bp, Artificial Sequence
  P1
gggtgttggctgatatgtgt

SEQ ID NO: 24 - DNA, 20 bp, Artificial Sequence
  P2
cctatgcctacagcatccag

SEQ ID NO: 25 - DNA, 20 bp, Artificial Sequence
  P3
acgtcccaggcatcaaataa

SEQ ID NO: 26 - DNA, 19 bp, Artificial Sequence
  P4
gaaggagcactctagcacg

SEQ ID NO: 27 - DNA, 20 bp, Artificial Sequence
  P5
tttttccaccacctcgatgc

SEQ ID NO: 28 - DNA, 19 bp, Artificial Sequence
  P6
gaattttcgcgaactcgcc

(19) United States
(12) Patent Application Publication
(10) Pub. No.: US 2021/0189387 A1
(43) Pub. Date: Jun. 24, 2021

(54) GENE EDITING SYSTEM OF PSEUDOMONAS PUTIDA AND GENE EDITING METHOD THEREOF

(71) Applicants: National Tsing Hua University, Hsinchu (TW); Chang Chun Plastics Co., Ltd., Taipei (TW); Chang Chun Petrochemical Co., Ltd., Taipei (TW); Dairen Chemical Corp., Taipei (TW)

(72) Inventors: Yu-Chen Hu, Hsinchu (TW); Nam Ngoc Pham, Hsinchu (TW); Cho-Yi Chen, Hsinchu (TW)

(21) Appl. No.: 17/077,030

(22) Filed: Oct. 22, 2020

(30) Foreign Application Priority Data
Dec. 20, 2019 (TW) 108147047

(51) Int. Cl. C12N 15/11; C12N 9/22

(52) U.S. Cl. CPC C12N 15/11; C12N 2800/80; C12N 2310/20; C12N 9/22

(57) ABSTRACT

The present disclosure relates to a gene editing system of Pseudomonas putida. The gene editing system of Pseudomonas putida includes a Pseudomonas putida, a RedCas expression plasmid and an exogenous gene expression plasmid. The RedCas expression plasmid includes a first replication origin, a first antibiotic resistance gene, a λ-Red expression cassette and a Cas expression cassette. The exogenous gene expression plasmid includes a second replication origin, a left homology arm, a second antibiotic resistance gene, an exogenous gene expression cassette, a right homology arm and a gRNA cassette.

### RELATED APPLICATIONS

This application claims priority to Taiwan Application Serial Number 108147047, filed Dec. 20, 2019, which is herein incorporated by reference.

### SEQUENCE LISTING

The sequence listing submitted via EFS, in compliance with 37 CFR § 1.52(e)(5), is incorporated herein by reference. The sequence listing text file submitted via EFS contains the file “CP-4599-US_SEQ_LIST”, created on Aug. 6, 2020, which is 35,663 bytes in size.

### BACKGROUND

### Technical Field

The present disclosure relates to a gene expression regulation system of Pseudomonas putida and an application thereof. More particularly, the present disclosure relates to a gene editing system of Pseudomonas putida and an application thereof.

### Description of Related Art

Pseudomonas putida is a Gram-negative, saprotrophic soil bacterium. Based on 16S rRNA analysis, Pseudomonas putida was taxonomically confirmed to be a Pseudomonas species.

Pseudomonas putida has high salt tolerance, can use glycerol as a carbon source for growth, and has many metabolic pathways that can decompose many organic molecules (such as degradable toluene). Thus, Pseudomonas putida is widely used in bioremediation technology, or for microbial decomposition of oil leakage, and can be used as an important strain platform for treating large amounts of salt-containing glycerin wastewater in the petrochemical industry. In addition, compared with other strains of Pseudomonas, Pseudomonas putida is safer and harmless, and unlike Pseudomonas aeruginosa has the possibility of becoming a human pathogen.

Conventional technology in the method of transforming exogenous gene into Pseudomonas putida for decomposition of organic molecules is still limited. It is still necessary to continue to transform plasmid and use antibiotics to maintain the stable existence of the exogenous gene. The strains that have completed the transformation are often unstable and easily lose the ability to decompose organic molecules, so that additional antibiotics are needed to maintain their ability to decompose organic molecules. The additional addition of antibiotics will add a lot of cost in industrial production. In addition to the aforementioned methods, there is reported that CRIM can be used to integrate exogenous genes into the chromosomes of Pseudomonas putida and perform gene editing, which requires repeated transformations with plasmid into the same chromosome site. However, the method is difficult to accurately integrate exogenous gene in the chromosome, and it is easy to lose the exogenous gene. Or the method is necessary to perform gene editing with a very large or complex plasmid transformation system, as the result, the gene editing process take nearly two weeks. Although there are other studies that use specific enzyme cleavage (I-SecI) cause double strand break (DSBs) to shorten the time for gene editing, the gene editing cannot be arbitrary due to the restriction of enzyme cleavage to certain site on the chromosome. Therefore, how to obtain a Pseudomonas putida transformant that stably expresses the exogenous gene for effectively applying in bioremediation to remove or neutralize contaminants in a contaminated site is a very important issue.

### SUMMARY

According to one aspect of the present disclosure, a gene editing system of Pseudomonas putida is provided. The gene editing system of Pseudomonas putida includes a Pseudomonas putida, a RedCas expression plasmid and an exogenous gene expression plasmid. The RedCas expression plasmid successively includes a first replication origin, a first antibiotic resistance gene, a A-Red expression cassette and a Cas expression cassette, in which the first replication origin includes a nucleic acid sequence of SEQ ID NO: 1, the A-Red expression cassette includes a first promoter, a Gam gene, a Bet gene and an Exo gene, and the Cas expression cassette includes a second promoter and a Cas gene. The exogenous gene expression plasmid successively includes a second replication origin, a left homology arm, a second antibiotic resistance gene, an exogenous gene expression cassette, a right homology arm and a gRNA cassette, in which the exogenous gene expression cassette includes a third promoter and an exogenous gene, the gRNA cassette includes a fourth promoter and a gRNA sequence, and the gRNA sequence is composed of a spacer and a scaffold. The left homology arm and the right homology arm compose a homology region. A sequence of the homology region is homologous to a first specific sequence of a chromosome of the Pseudomonas putida, and a sequence of the spacer is homologous to a second specific sequence of the chromosome of the Pseudomonas putida. The first antibiotic resistance gene and the second antibiotic resistance gene are different.

According to another aspect of the present disclosure, a gene editing method of Pseudomonas putida includes steps as follows. A RedCas expression plasmid is constructed. The RedCas expression plasmid successively includes a first replication origin, a first antibiotic resistance gene, a A-Red expression cassette and a Cas expression cassette, in which the first replication origin includes a nucleic acid sequence of SEQ ID NO: 1, the A-Red expression cassette includes a first promoter, a Gam gene, a Bet gene and an Exo gene, and the Cas expression cassette includes a second promoter and a Cas gene. An exogenous gene expression plasmid is constructed. The exogenous gene expression plasmid successively includes a second replication origin, a left homology arm, a second antibiotic resistance gene, an exogenous gene expression cassette, a right homology arm and a gRNA cassette, in which the exogenous gene expression cassette includes a third promoter and an exogenous gene, the gRNA cassette includes a fourth promoter and a gRNA sequence composed of a spacer and a scaffold. The left homology arm and the right homology arm compose a homology region, a sequence of the homology region is homologous to a first specific sequence of a chromosome of a Pseudomonas putida, a sequence of the spacer is homologous to a second specific sequence of the chromosome of the Pseudomonas putida, and the first antibiotic resistance gene and the second antibiotic resistance gene are different. The RedCas expression plasmid is transformed into the Pseudomonas putida to obtain a first transformant. An induction step is performed, in which the first transformant is cultured and then an inducer is added to induce the RedCas expression plasmid expresses a Gam protein, a Beta protein, an Exo protein and a Cas protein to obtain an induced first transformant. The exogenous gene expression plasmid is transformed into the induced first transformant to obtain a second transformant, so that the exogenous gene expression plasmid expresses a gRNA. The gRNA and the Cas protein form a Cas protein complex triggering a double strand break on the second specific sequence of the chromosome of the second transformant, and the Gam protein, the Beta protein and the Exo protein co-guide the homology region of the exogenous gene expression plasmid to perform homologous recombination with the first specific sequence of the chromosome of the second transformant, so that the exogenous gene is integrated into the first specific sequence of the chromosome of the second transformant.

### BRIEF DESCRIPTION OF THE DRAWINGS

The present disclosure can be more fully understood by reading the following detailed description of the embodiment, with reference made to the accompanying drawings as follows:

FIG. 1A is a schematic view showing a construction of a RedCas expression plasmid according to one embodiment of the present disclosure.

FIG. 1B is a schematic view showing a construction of an exogenous gene expression plasmid according to one embodiment of the present disclosure.

FIG. 2 is a flow diagram showing a gene editing method of Pseudomonas putida according to another embodiment of the present disclosure.

FIG. 3A is a schematic view showing constructions of test examples of CRISPR/Cas system.

FIG. 3B is a qualitative diagram of double strand break efficiency of Pseudomonas putida triggered by the CRISPR/Cas system.

FIG. 3C is a quantitative diagram of double strand break efficiency of Pseudomonas putida triggered by the CRISPR/Cas system.

FIG. 4A is a schematic view showing constructions of Example 1, Example 2 and Example 3 of the RedCas expression plasmid according to one embodiment of the present disclosure.

FIG. 4B is a schematic view showing constructions of Example 1, Example 2 and Example 3 of the exogenous gene expression plasmid according to one embodiment of the present disclosure.

FIG. 4C is a schematic view showing operation of a gene editing system of Pseudomonas putida of the present disclosure in the Pseudomonas putida.

FIG. 4D is a schematic view showing constructions of Comparative Example 1, Comparative Example 2 and Comparative Example 3 of the gene editing system of Pseudomonas putida.

FIG. 4E is a schematic view showing constructions of Comparative Example 4, Comparative Example 5 and Comparative Example 6 of the gene editing system of Pseudomonas putida.

FIG. 5A is a qualitative diagram of survival colonies of the Pseudomonas putida treated with Example 1, Example 2 and Example 3 of the gene editing system of Pseudomonas putida of the present disclosure.

FIG. 5B is a qualitative diagram of survival colonies of the Pseudomonas putida treated with Comparative Example 1, Comparative Example 2 and Comparative Example 3 of the gene editing system of Pseudomonas putida.

FIG. 5C is a qualitative diagram of survival colonies of the Pseudomonas putida treated with Comparative Example 4, Comparative Example 5 and Comparative Example 6 of the gene editing system of Pseudomonas putida.

FIG. 5D shows analytical results of the homologous recombination efficiency of Example 1, Example 2 and Example 3 of the gene editing system of Pseudomonas putida of the present disclosure.

FIG. 5E is a quantitative diagram of the percentage of survival colonies of the Pseudomonas putida treated with Example 1, Example 2 and Example 3 of the gene editing system of Pseudomonas putida of the present disclosure.

FIG. 6A is a schematic diagram showing the sites of 6 pairs of primers designed for colony PCR.

FIGS. 6B, 6C and 6D show analytical results of the colony PCR for confirming the integration of the exogenous gene in the chromosome of the Pseudomonas putida.

FIG. 6E shows analytical result of the colony PCR for confirming the existence of the original chromosome sequence of the Pseudomonas putida after treating by the gene editing system of Pseudomonas putida.

FIG. 7A shows analytical results of the colony PCR for confirming integration of the exogenous gene in the chromosome of the Pseudomonas putida after 6 days of treatment by the gene editing system of Pseudomonas putida.

FIG. 7B is an analytical result showing gene copy number of the chromosome of the Pseudomonas putida which is integrated the exogenous gene.

### DETAILED DESCRIPTION

### Gene Editing System of Pseudomonas putida

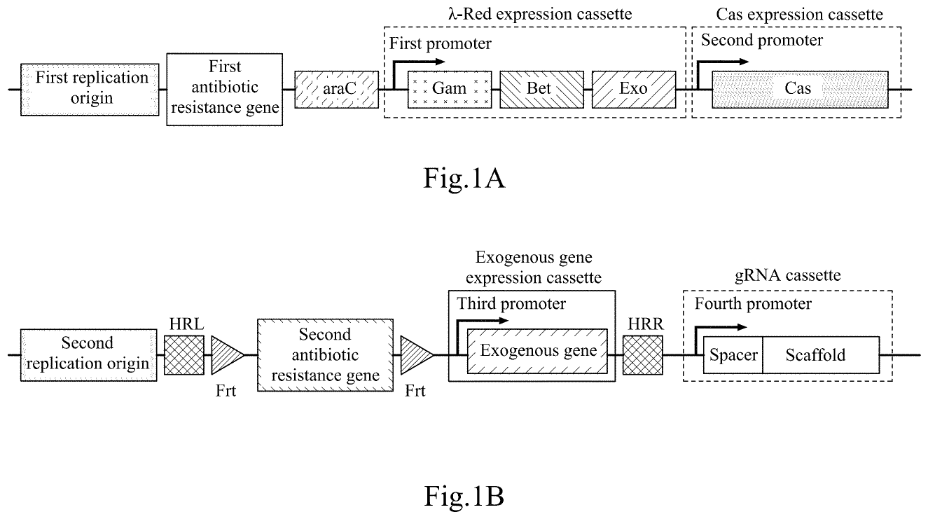

A gene editing system of Pseudomonas putida of the present disclosure includes a Pseudomonas putida, a RedCas expression plasmid and an exogenous gene expression plasmid. Please refer to FIGS. 1A and 1B, FIG. 1A is a schematic view showing a construction of a RedCas expression plasmid according to one embodiment of the present disclosure, and FIG. 1B is a schematic view showing a construction of an exogenous gene expression plasmid according to one embodiment of the present disclosure.

The RedCas expression plasmid successively includes a first replication origin, a first antibiotic resistance gene, a A-Red expression cassette and a Cas expression cassette, in which the first replication origin includes a nucleic acid sequence of SEQ ID NO: 1, the A-Red expression cassette includes a first promoter, a Gam gene, a Bet gene and an Exo gene, and the Cas expression cassette includes a second promoter and a Cas gene. Preferably, the RedCas expression plasmid can further include an araC gene, and the first promoter can be an araBAD promoter. The first antibiotic resistance gene can be gentamicin resistance (GmR), kanamycin resistance (KmR) or tetracycline resistance (Tc^{R}).

The exogenous gene expression plasmid successively includes a second replication origin, a left homology arm, a second antibiotic resistance gene, an exogenous gene expression cassette, a right homology arm and a gRNA cassette, in which the exogenous gene expression cassette includes a third promoter and an exogenous gene, the gRNA cassette includes a fourth promoter and a gRNA sequence, and the gRNA sequence is composed of a spacer and a scaffold. The left homology arm and the right homology arm compose a homology region. A sequence of the homology region is homologous to a first specific sequence of a chromosome of the Pseudomonas putida, and a sequence of the spacer is homologous to a second specific sequence of the chromosome of the Pseudomonas putida. The first antibiotic resistance gene and the second antibiotic resistance gene are different. Preferably, the exogenous gene expression plasmid can further include two Flp/FRT knockout sequences, and the second antibiotic resistance gene is located between the two Flp/FRT knockout sequences. The second antibiotic resistance gene can be gentamicin resistance (GmR) gene, kanamycin resistance (KmR) gene or tetracycline resistance (Tc^{R}) gene.

### Gene Editing Method of Pseudomonas putida

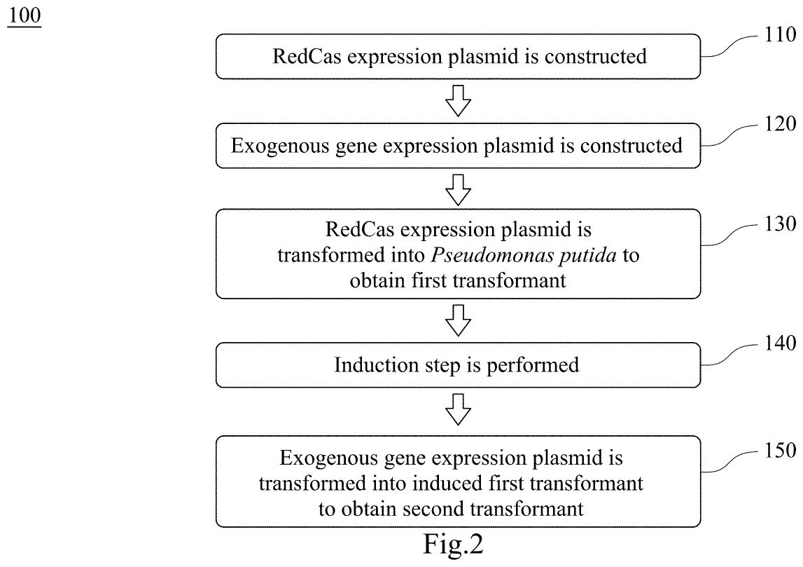

Please refer to FIG. 2, which is a flow diagram showing a gene editing method of Pseudomonas putida 100 according to another embodiment of the present disclosure. In FIG. 2, the gene editing method of Pseudomonas putida 100 includes a Step 110, a Step 120, a Step 130, a Step 140, and a Step 150.

In the Step 110, a RedCas expression plasmid is constructed. The RedCas expression plasmid successively includes a first replication origin, a first antibiotic resistance gene, a λ-Red expression cassette and a Cas expression cassette, in which the first replication origin includes a nucleic acid sequence of SEQ ID NO: 1, the λ-Red expression cassette includes a first promoter, a Gam gene, a Bet gene and an Exo gene, and the Cas expression cassette includes a second promoter and a Cas gene. Preferably, the RedCas expression plasmid can further include an araC gene, and the first promoter can be an araBAD promoter.

In the Step 120, an exogenous gene expression plasmid is constructed. The exogenous gene expression plasmid successively includes a second replication origin, a left homology arm, a second antibiotic resistance gene, an exogenous gene expression cassette, a right homology arm and a gRNA cassette, in which the exogenous gene expression cassette includes a third promoter and an exogenous gene, the gRNA cassette includes a fourth promoter and a gRNA sequence composed of a spacer and a scaffold, the left homology arm and the right homology arm compose a homology region, a sequence of the homology region is homologous to a first specific sequence of a chromosome of the Pseudomonas putida, a sequence of the spacer is homologous to a second specific sequence of the chromosome of the Pseudomonas putida, and the first antibiotic resistance gene and the second antibiotic resistance gene are different.

In the Step 130, the RedCas expression plasmid is transformed into the Pseudomonas putida to obtain a first transformant. Preferably, a first selection step can be further included, in which the first transformant is cultured in a first medium containing a first antibiotic to select the first transformant that successfully transformed the RedCas expression plasmid. The first antibiotic can be gentamicin, kanamycin or tetracycline.

In the Step 140, an induction step is performed. The first transformant is cultured and then an inducer is added to induce the RedCas expression plasmid expresses a Gam protein, a Beta protein, an Exo protein and a Cas protein to obtain an induced first transformant. Preferably, the inducer can be added after the first transformant is cultured to a stationary phase, and subsequent experiments are conducted. For example, adding the inducer after cultivating the first transformed strain for 12 hours can ensure the transformation efficiency.

In the Step 150, the exogenous gene expression plasmid is transformed into the induced first transformant to obtain a second transformant, so that the exogenous gene expression plasmid expresses a gRNA. The gRNA and the Cas protein form a Cas protein complex triggering a double strand break on the second specific sequence of the chromosome of the second transformant, and the Gam protein, the Beta protein and the Exo protein co-guide the homology region of the exogenous gene expression plasmid to perform homologous recombination with the first specific sequence of the chromosome of the second transformant, so that the exogenous gene is integrated into the first specific sequence of the chromosome of the second transformant. Preferably, a second selection step can be further included, in which the second transformant is cultured in a second medium containing a second antibiotic to select the second transformant that successfully transformed the exogenous gene expression plasmid. The second antibiotic can be gentamicin, kanamycin or tetracycline.

Reference will now be made in detail to the present embodiments of the present disclosure, examples of which are illustrated in the accompanying drawings.

### 1. Establishment of a CRISPR/Cas System of Pseudomonas putida

To date, whether the CRISPR/Cas can be applied to Pseudomonas putida is still unknown. In order to verify that the CRISPR/Cas system can trigger double strand breaks in Pseudomonas putida, three different CRISPR/Cas systems are constructed, including SpCas9, SaCas9, and FnCas12a, and gRNA corresponds to the Upp site of the chromosome of Pseudomonas putida (PP_0746) is designed as Test Example, respectively. Pseudomonas putida used in the experiment is Pseudomonas putida S12.

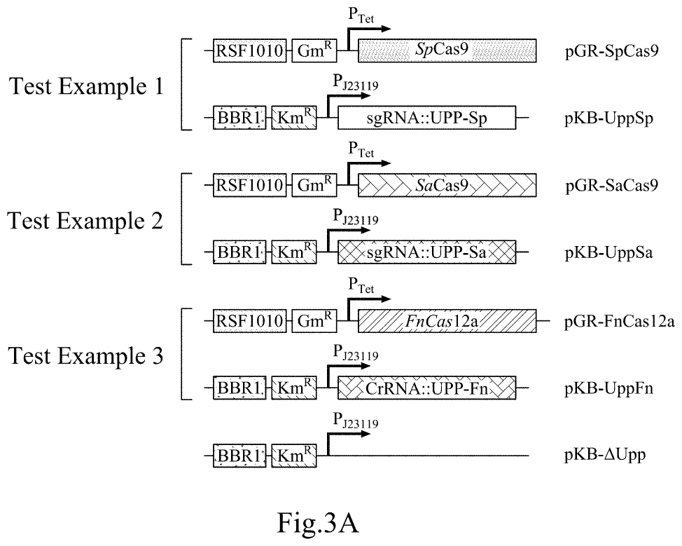

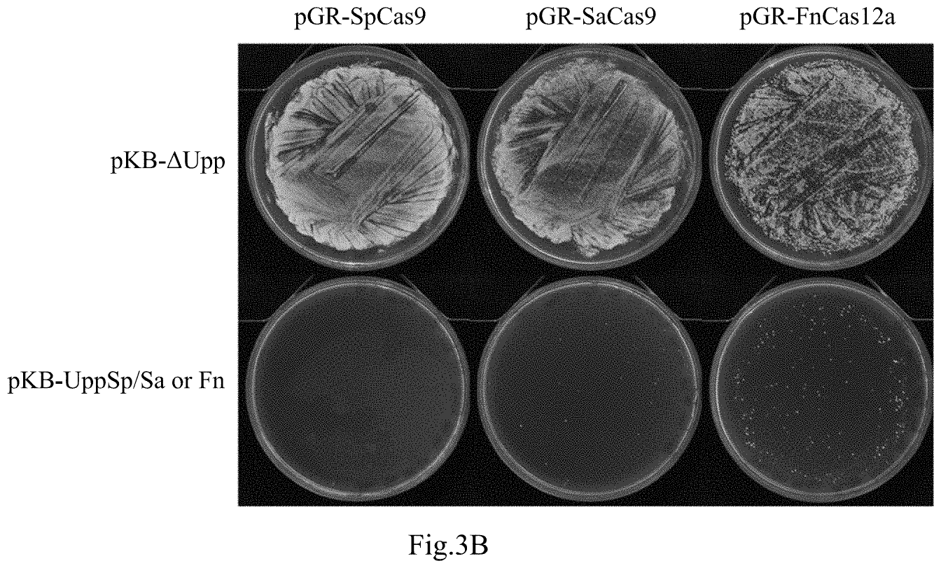

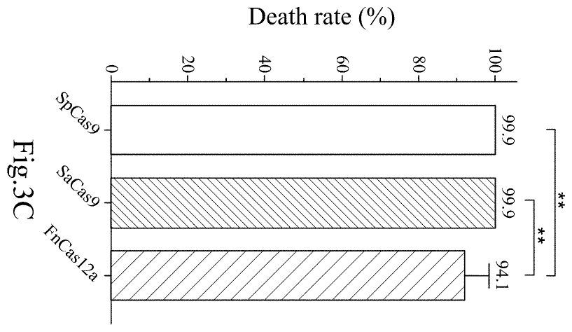

Please refer to FIG. 3A, which is a schematic view showing constructions of Test Examples of CRISPR/Cas system. Test Example 1 includes pGR-SpCas9 plasmid and pKB-UppSp plasmid. The pGR-SpCas9 plasmid includes SpCas9 gene with the nucleotide sequence referenced as SEQ ID NO: 9. The pKB-UppSp plasmid includes the gRNA sequence corresponding to the Upp site of the chromosome of Pseudomonas putida with the nucleotide sequence referenced as SEQ ID NO: 18. Test Example 2 includes pGR-SaCas9 plasmid and pKB-UppSa plasmid. The pGR-SaCas9 plasmid includes SaCas9 gene with the nucleotide sequence referenced as SEQ ID NO: 10. The pKB-UppSa plasmid includes the gRNA sequence corresponding to the Upp site of the chromosome of Pseudomonas putida with the nucleotide sequence referenced as SEQ ID NO: 19. Test Example 3 includes pGR-FnCas12a plasmid and pKB-UppFn plasmid. The pGR-FnCas12a plasmid includes FnCas12a gene with the nucleotide sequence referenced as SEQ ID NO: 11. The pKB-UppFn plasmid includes the gRNA sequence corresponding to the Upp site of the chromosome of Pseudomonas putida with the nucleotide sequence referenced as SEQ ID NO: 20. In addition, in order to verify the efficiency of double strand break in different systems, pKB-ΔUpp plasmid is constructed as a control group in the experiment, which will not trigger double strand break on the chromosome of Pseudomonas putida.

To confirm that the CRISPR/Cas system is indeed effective in Pseudomonas putida, the constructed plasmid is transformed into the Pseudomonas putida, and then the expected double strand break at the target site on the chromosome of Pseudomonas putida triggered by the Cas protein complex is observed. If the Cas protein complex successfully triggers the double strand break, the Pseudomonas putida will die as a result. The number of survival colonies can verify whether the CRISPR/Cas system is effective and whether the CRISPR/Cas system will cause toxic effects on Pseudomonas putida. The experiment includes experimental groups and control groups. In the experimental groups, Test Example 1, Test Example 2 or Test Example 3 is transformed into the Pseudomonas putida. In the control groups, the pKB-UppSp plasmid in Test Example 1, pKB-UppSa plasmid in Test Example 2 and pKB-UppFn plasmid in Test Example 3 are replaced with pKB-ΔUpp plasmid. The efficiency of the CRISPR/Cas system to trigger double strand break in Pseudomonas putida is verified by the death rate of Pseudomonas putida. The death rate is calculated by the following formula I, wherein CFUₑₓₚₑᵣᵢₘₑₙₜₐₗ represents colony numbers of the experimental group, and CFU_{control} represents colony numbers of the control group.

$$Death rate ( \% ) = [ 1 - \frac{{CFU}_{experimental}}{{CFU}_{control}} ] \times 100. \qquad formula 1$$

Please refer to FIGS. 3B and 3C, FIG. 3B is a qualitative diagram of double strand break efficiency of Pseudomonas putida triggered by the CRISPR/Cas system, and FIG. 3C is a quantitative diagram of double strand break efficiency of Pseudomonas putida triggered by the CRISPR/Cas system. The results show that the death rate caused by Test Example 1 and Test Example 2 are both 99.9%, and the death rate caused by Test Example 3 is 94.1%. It is speculated that the DNA gap after double strand break triggered by FnCas12a is uneven, and the DNA gap after double strand break triggered by SpCas9 and SaCas9 is flat. The damage caused by FnCas12a is more likely to be repaired by Pseudomonas putida. Therefore, the death rate of Pseudomonas putida eventually showed a significant difference.

### 2. Establishment of a Gene Editing System of Pseudomonas putida

After confirming that the CRISPR/Cas system can indeed trigger the double strand break of Pseudomonas putida, three different gene editing systems of Pseudomonas putida by SpCas9, SaCas9 and FnCas12a are constructed. The gene editing system of Pseudomonas putida of the present disclosure includes the Pseudomonas putida, the RedCas expression plasmid and the exogenous gene expression plasmid.

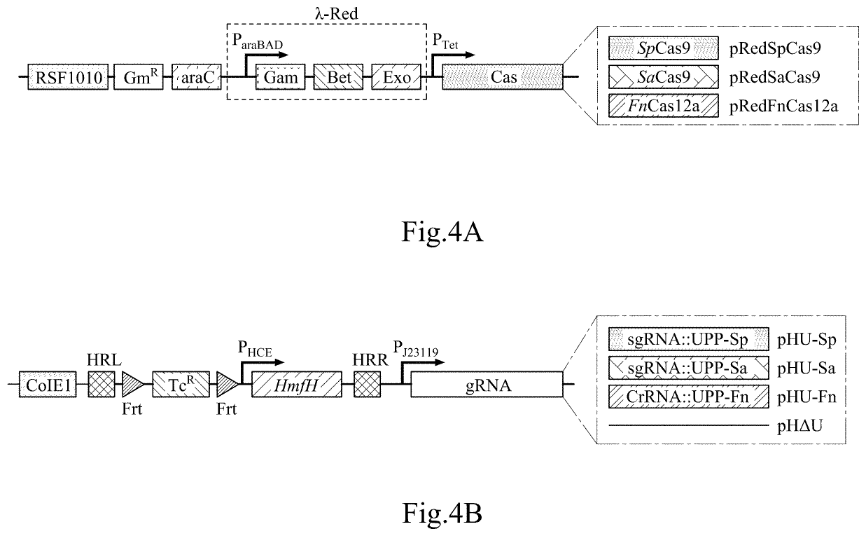

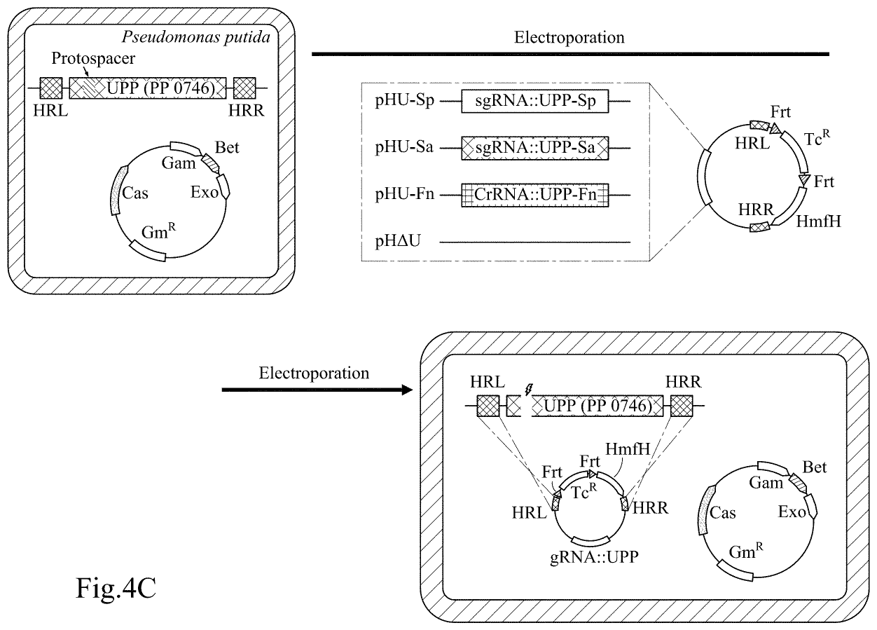

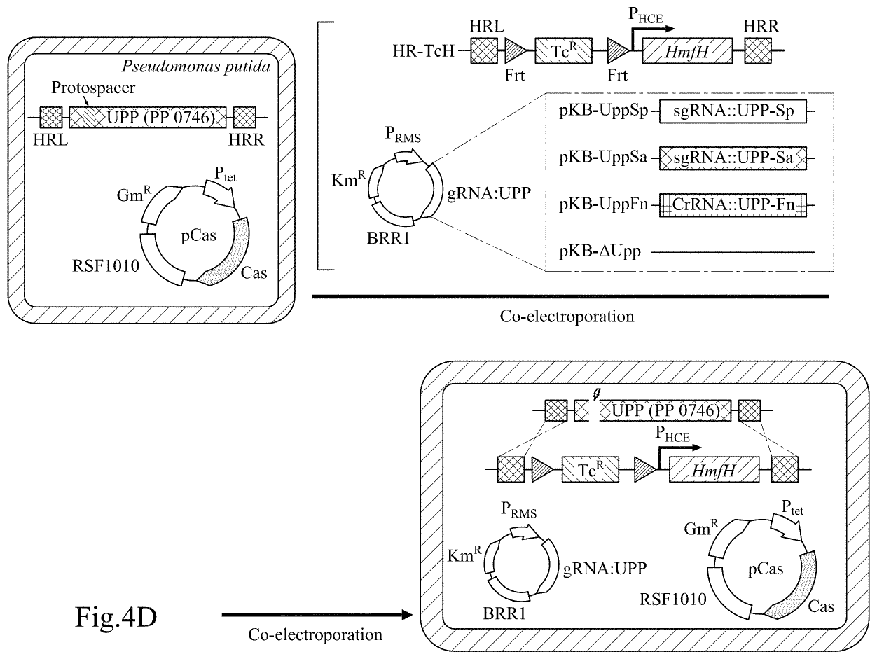

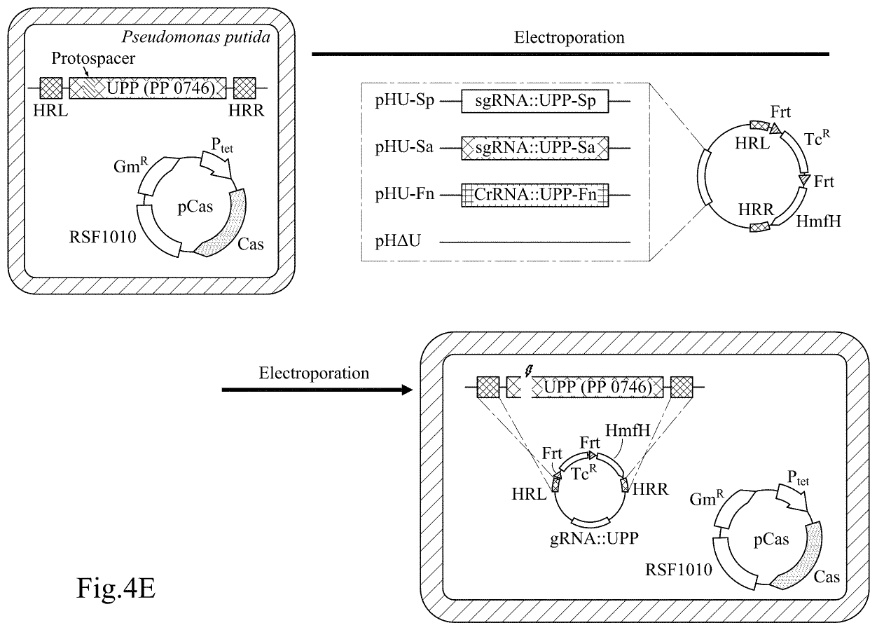

Please refer to FIG. 4A, which is a schematic view showing constructions of Example 1, Example 2 and Example 3 of the RedCas expression plasmid according to one embodiment of the present disclosure. According to one example of this embodiment, the RedCas expression plasmid successively includes the first replication origin, the first antibiotic resistance gene, the araC gene, the λ-Red expression cassette and the Cas expression cassette, wherein the first replication origin is RSF1010 ori with the nucleotide sequence referenced as SEQ ID NO: 1, the first antibiotic resistance gene is gentamicin resistance (GmR) gene with the nucleotide sequence referenced as SEQ ID NO: 2, the nucleotide sequence of the araC gene is referenced as SEQ ID NO: 3. The λ-Red expression cassette includes the first promoter, the Gam gene, the Bet gene and the Exo gene, wherein the first promoter is araBAD promoter with the nucleotide sequence referenced as SEQ ID NO: 4, the nucleotide sequence of the Gam gene is referenced as SEQ ID NO: 5, the nucleotide sequence of the Bet gene is referenced as SEQ ID NO: 6, and the nucleotide sequence of the Exo gene is referenced as SEQ ID NO: 7. The Cas expression cassette includes the second promoter and the Cas gene, wherein the second promoter is Tet promoter with the nucleotide sequence referenced as SEQ ID NO: 8. The RedCas expression plasmids of the present disclosure include Example 1, Example 2 and Example 3, which are pRedSpCas9 plasmid, pRedSaCas9 plasmid and pRedFnCas12a plasmid, respectively. The nucleotide sequence of the Cas gene of the pRedSpCas9 plasmid is referenced as SEQ ID NO: 9, the nucleotide sequence of the Cas gene of the pRedSaCas9 plasmid is referenced as SEQ ID NO: 10, and the nucleotide sequence of the Cas gene of the pRedFnCas12a plasmid is referenced as SEQ ID NO: 11.

Please refer to FIG. 4B, which is a schematic view showing constructions of Example 1, Example 2 and Example 3 of the exogenous gene expression plasmid according to one embodiment of the present disclosure. According to one example of this embodiment, the exogenous gene expression plasmid successively includes the second replication origin, the left homology arm, the Flp/FRT knockout sequence, the second antibiotic resistance gene, the Flp/FRT knockout sequence, the exogenous gene expression cassette, the right homology arm and the gRNA cassette. The second replication origin is ColE1 ori with the nucleotide sequence referenced as SEQ ID NO: 12, the nucleotide sequence of the left homology arm is referenced as SEQ ID NO: 15, the nucleotide sequence of the Flp/FRT knockout sequence is referenced as SEQ ID NO: 22, and the second antibiotic resistance gene is tetracycline resistance (Tc^{R}) gene with the nucleotide sequence referenced as SEQ ID NO: 13. The exogenous gene expression cassette includes the third promoter and the exogenous gene, wherein the third promoter is HCE promoter with the nucleotide sequence referenced as SEQ ID NO: 14, and the exogenous gene is HmfH gene as an example with the nucleotide sequence referenced as SEQ ID NO: 21. The nucleotide sequence of the right homology arm is referenced as SEQ ID NO: 16. The gRNA cassette includes the fourth promoter and the gRNA sequence, wherein the gRNA sequence is composed of the spacer and the scaffold. The fourth promoter is J23119 promoter with the nucleotide sequence referenced as SEQ ID NO: 17. The exogenous gene expression plasmids of the present disclosure include Example 1, Example 2 and Example 3, which are pHU-Sp plasmid, pHU-Sa plasmid and pHU-Fn plasmid, respectively. The nucleotide sequence of the gRNA sequence of the pHU-Sp plasmid is referenced as SEQ ID NO: 18, the nucleotide sequence of the gRNA sequence of the pHU-Sa plasmid is referenced as SEQ ID NO: 19, and the nucleotide sequence of the gRNA sequence of the pHU-Fn plasmid is referenced as SEQ ID NO: 20. In addition, pHΔU plasmid is constructed as a control group, which does not trigger double strand break on the chromosome of Pseudomonas putida. The left homology arm and the right homology arm compose the homology region. The sequence of the homology region is homologous to the first specific sequence of the chromosome of the Pseudomonas putida, the sequence of the spacer is homologous to Upp site of the chromosome of the Pseudomonas putida, and the scaffold interacts with the Cas protein.

Please refer to FIG. 4C, which is a schematic view showing operation of the gene editing system of Pseudomonas putida of the present disclosure in the Pseudomonas putida. The constructed RedCas expression plasmid (pRedSpCas9 plasmid, pRedSaCas9 plasmid, and pRedFnCas12a plasmid) is transformed into Pseudomonas putida by electroporation to obtain the first transformant, respectively. Further, the first transformant can be cultured using LB medium containing gentamicin to select the first transformant that successfully transformed the RedCas expression plasmid. The first transformant is cultured in 30 mL of fresh LB medium containing gentamicin overnight, and then cultured with 0.5% arabinose for another 2 hours to induce the RedCas expression plasmid expresses the Gam protein, the Beta protein, the Exo protein and the Cas protein. The inducted first transformant is collected by centrifugation and washed twice with 300 mM sucrose. Then, the exogenous gene expression plasmid (pHU-Sp plasmid, pHU-Sa plasmid and pHU-Fn plasmid) is transformed into the corresponding first transformant by electroporation to obtain the second transformant, respectively. Further, the second transformant can be cultured with LB medium containing tetracycline to select the second transformant that successfully transformed the exogenous gene expression plasmid. The second transformant is cultured, so that the exogenous gene expression plasmid expresses the gRNA. The gRNA and the Cas protein form a Cas protein complex triggering the double strand break on the second specific sequence of the chromosome of the second transformant, and the Gam protein, the Beta protein and the Exo protein co-guide the homology region of the exogenous gene expression plasmid to perform homologous recombination with the first specific sequence of the chromosome of the second transformant, so that the HmfH gene is integrated into the Upp site of the chromosome of the second transformant to obtain the homologous recombinant.

Furthermore, the CRISPR system is used to construct Comparative Examples 1-6 of the gene editing system of Pseudomonas putida. Please refer to FIGS. 4D and 4E, FIG. 4D is a schematic view showing constructions of Comparative Example 1, Comparative Example 2 and Comparative Example 3 of the gene editing system of Pseudomonas putida, FIG. 4E is a schematic view showing constructions of Comparative Example 4, Comparative Example 5 and Comparative Example 6 of the gene editing system of Pseudomonas putida.

As shown in FIG. 4D, in Comparative Example 1, Comparative Example 2 and Comparative Example 3, the gRNA sequence and the exogenous gene are constructed in gRNA transcript plasmid and template plasmid, respectively. The template plasmids of Comparative Example 1, Comparative Example 2 and Comparative Example 3 are the same, including the left homology arm (length about 1000 bps), the Flp/FRT knockout sequence, the tetracycline resistance (Tc^{R}) gene, the Flp/FRT knockout sequence, the HCE promoter and the HmfH gene. The gRNA transcript plasmid of Comparative Example 1, Comparative Example 2 and Comparative Example 3 are pKB-UppSp plasmid, pKB-UppSa plasmid, and pKB-UppFn plasmid, respectively. The nucleotide sequence of the gRNA sequence of the pKB-UppSp plasmid is referenced as SEQ ID NO: 18, the nucleotide sequence of the gRNA sequence of the pKB-UppSa plasmid is referenced as SEQ ID NO: 19, and the nucleotide sequence of the gRNA sequence of the pKB-UppFn plasmid is referenced as SEQ ID NO: 20. In addition, pKB-ΔUpp plasmid is constructed as a control group, which does not trigger double strand break on the chromosome of Pseudomonas putida. In Comparative Example 1, Comparative Example 2 and Comparative Example 3 of the gene editing system of Pseudomonas putida, cells with Cas protein expressing plasmid (pGR-SpCas9, pGR-SaCas9, or pGR-FnCas12a) is used as competent cell, and then the template plasmid and the corresponding gRNA transcript plasmid (pKB-UppSp plasmid, pKB-UppSa plasmid, pKB-UppFn plasmid or pKB-ΔUpp plasmid) are co-transformed into the competent cell by electroporation. Then, the co-transformed cells are plated on a solid medium containing tetracycline for qualitative analysis of survival colonies.

As shown in FIG. 4E, in Comparative Example 4, Comparative Example 5 and Comparative Example 6, both of the gRNA sequence and the exogenous gene are constructed in the exogenous gene expression plasmid. The exogenous gene expression plasmid of Comparative Example 4 is pHU-Sp plasmid (same as the exogenous gene expression plasmid of Example 1), the exogenous gene expression plasmid of Comparative Example 5 is pHU-Sa plasmid (same as the exogenous gene expression plasmid of Example 2), and the exogenous gene expression plasmid of Comparative Example 6 is pHU-Fn plasmid (same as the exogenous gene expression plasmid of Example 3). In addition, the pHΔU plasmid is constructed as a control group, which does not trigger double strand break on the chromosome of Pseudomonas putida. In Comparative Example 4, Comparative Example 5 and Comparative Example 6 of the gene editing system of Pseudomonas putida, cells with Cas protein expressing plasmid (pGR-SpCas9, pGR-SaCas9, or pGR-FnCas12a) is used as competent cell, and then the corresponding exogenous gene expression plasmid (pHU-Sp plasmid, pHU-Sa plasmid, pHU-Fn plasmid or pHΔU plasmid) is transformed into the competent cell by electroporation. Then, the transformed cells are plated on a solid medium containing tetracycline for qualitative analysis of survival colonies.

To confirm that the gene editing system of Pseudomonas putida of the present disclosure is indeed effective in Pseudomonas putida, after transforming the constructed plasmid into the Pseudomonas putida, the obtained second transformant is selected by culturing in the medium containing tetracycline. The number of survival colonies represents the number of second transformant that homologously recombine the exogenous gene into the chromosome of the first transformant.

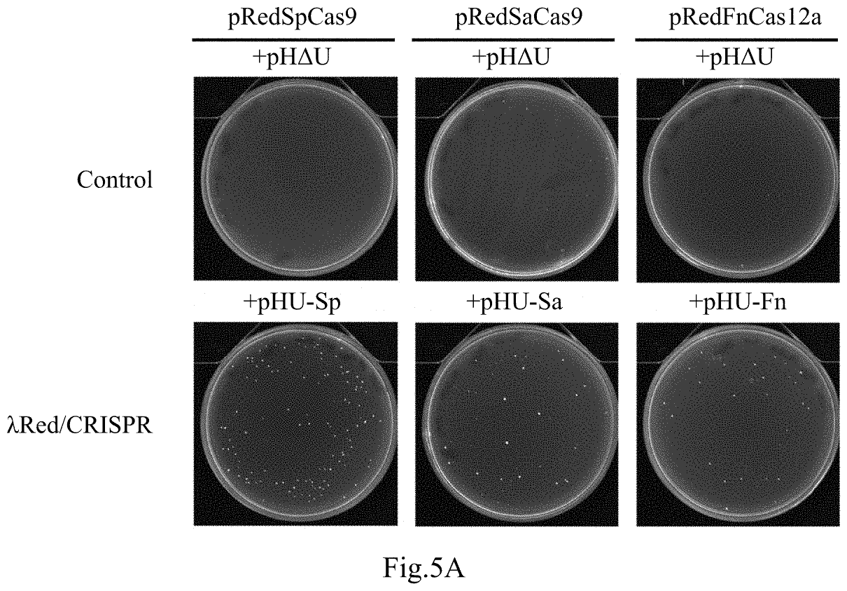

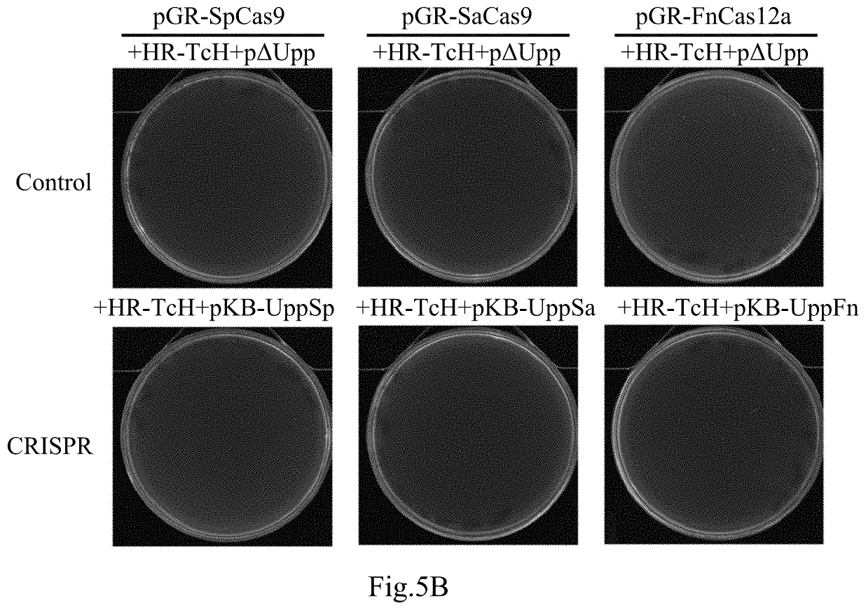

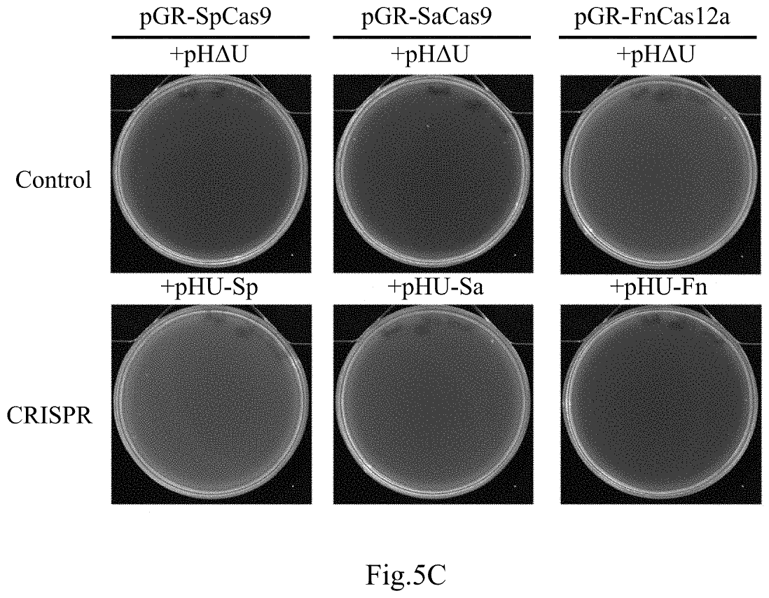

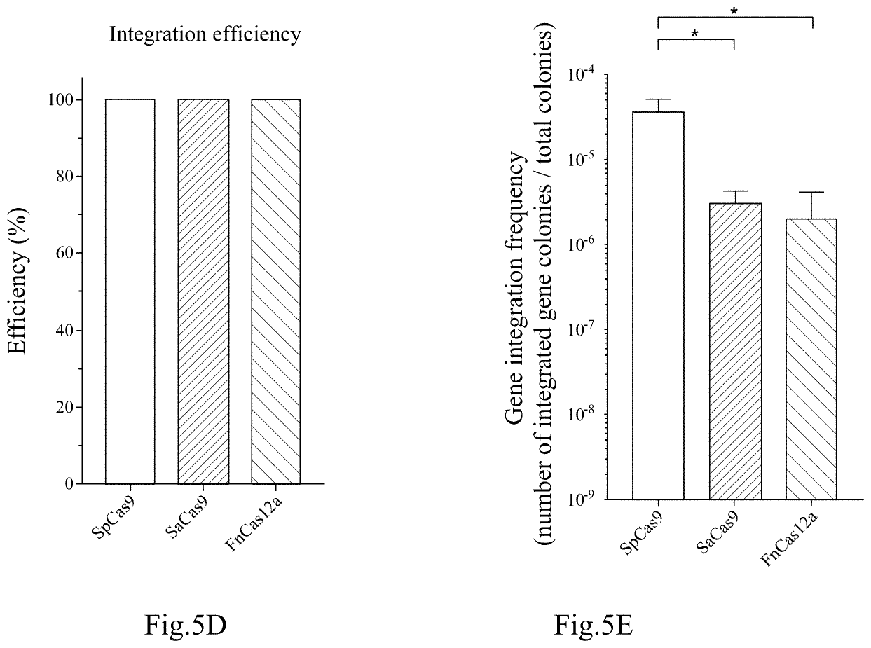

Please refer to FIGS. 5A, 5B, 5C, 5D and 5E. FIG. 5A is a qualitative diagram of survival colonies of the Pseudomonas putida treated with Example 1, Example 2 and Example 3 of the gene editing system of Pseudomonas putida of the present disclosure. FIG. 5B is a qualitative diagram of survival colonies of the Pseudomonas putida treated with Comparative Example 1, Comparative Example 2 and Comparative Example 3 of the gene editing system of Pseudomonas putida. FIG. 5C is a qualitative diagram of survival colonies of the Pseudomonas putida treated with Comparative Example 4, Comparative Example 5 and Comparative Example 6 of the gene editing system of Pseudomonas putida. FIG. 5D shows analytical results of the homologous recombination efficiency of Example 1, Example 2 and Example 3 of the gene editing system of Pseudomonas putida of the present disclosure. FIG. 5E is a quantitative diagram of the percentage of survival colonies of the Pseudomonas putida treated with Example 1, Example 2 and Example 3 of the gene editing system of Pseudomonas putida of the present disclosure.

In FIG. 5A, survival colonies can be obviously observed in the groups with gRNA (pHU), but no survival colony can be observed in the groups without gRNA (pHΔU). The results indicate that the λ-Red system alone cannot effectively integrate the exogenous gene into the chromosome of Pseudomonas putida without the double strand break triggered by the CRISPR system, so that the integration efficiency of the exogenous gene is very low. In addition, the survival rate of the gene edited Pseudomonas putida is calculated by taking the total number of the survival colonies without antibiotic as the denominator, and the number of the survival colonies with antibiotic (that is the number of the colonies successfully integrate the exogenous gene) as the numerator. In FIGS. 5B and 5C, there is no survival colony in the Pseudomonas putida treated with Comparative Example 1, Comparative Example 2, Comparative Example 3, Comparative Example 4, Comparative Example 5 or Comparative Example 6 of the gene editing system of Pseudomonas putida. The results indicate that the gene editing system of Pseudomonas putida using the CRISPR system alone cannot effectively integrate the exogenous gene into the chromosome of Pseudomonas putida. In FIGS. 5D and 5E, the integration efficiency of the exogenous gene in the Pseudomonas putida treated with Example 1, Example 2 or Example 3 the gene editing system of Pseudomonas putida can reach nearly 100%, and the survival rate of SpCas9 system is higher than that of SaCas9 and FnCas12a.

The above results indicate that the gene editing system of the Pseudomonas putida with the λ-Red system or CRISPR system alone cannot effectively integrate the exogenous gene into the chromosomes of Pseudomonas putida. The gene editing system of Pseudomonas putida and method thereof of the present disclosure, which incorporate the λ-Red system and the CRISPR system, can successfully integrate the exogenous gene into the chromosomes of Pseudomonas putida.

### 3. The Gene Editing System of Pseudomonas putida Promotes a Homologous Recombination in the Pseudomonas putida

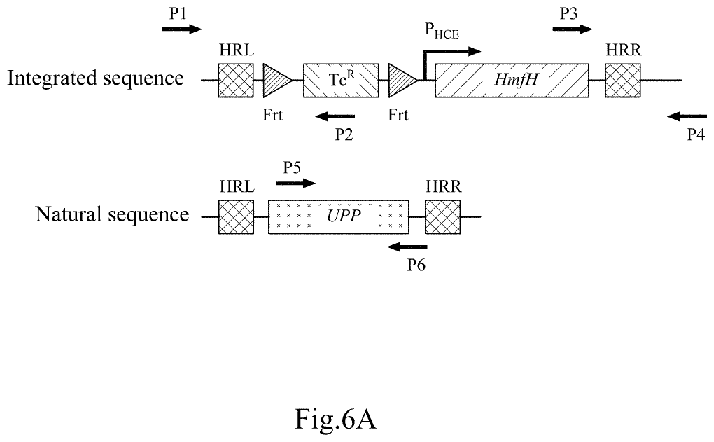

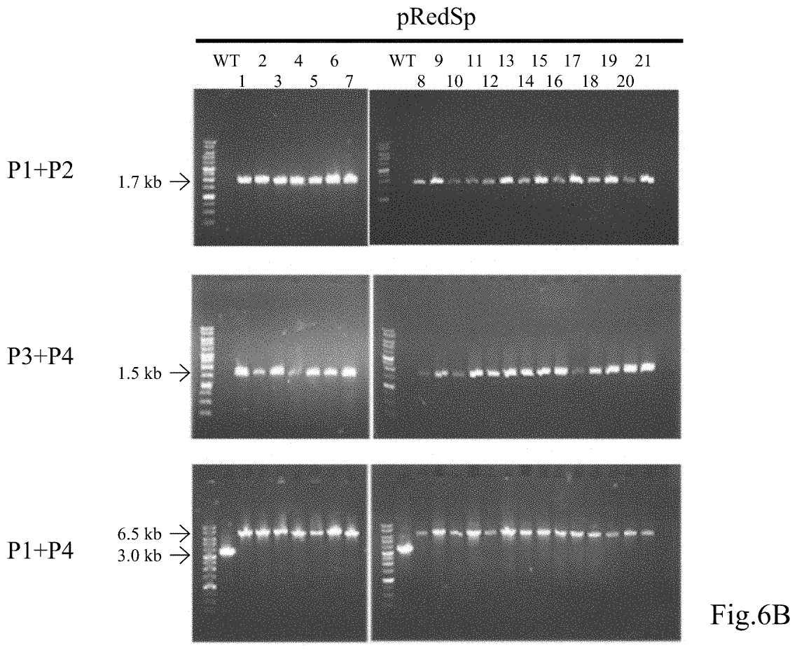

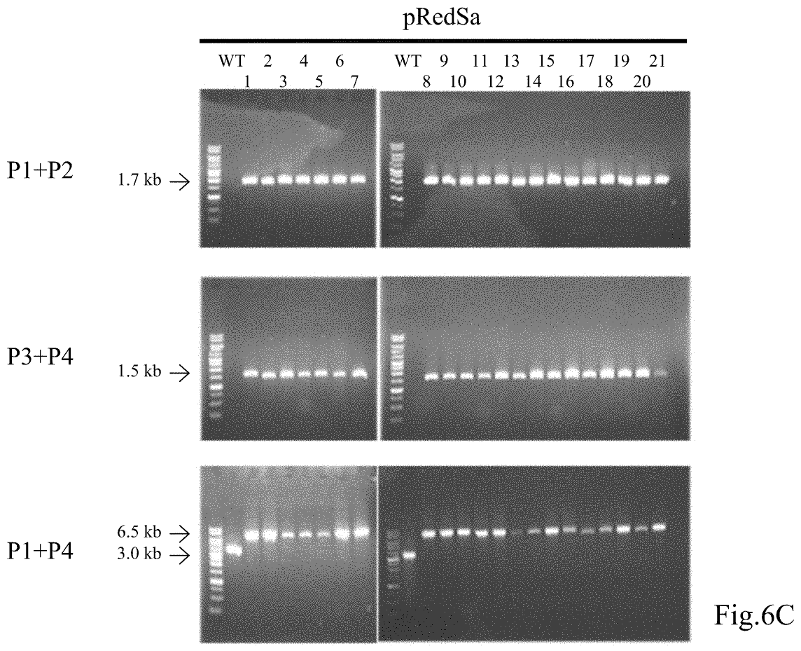

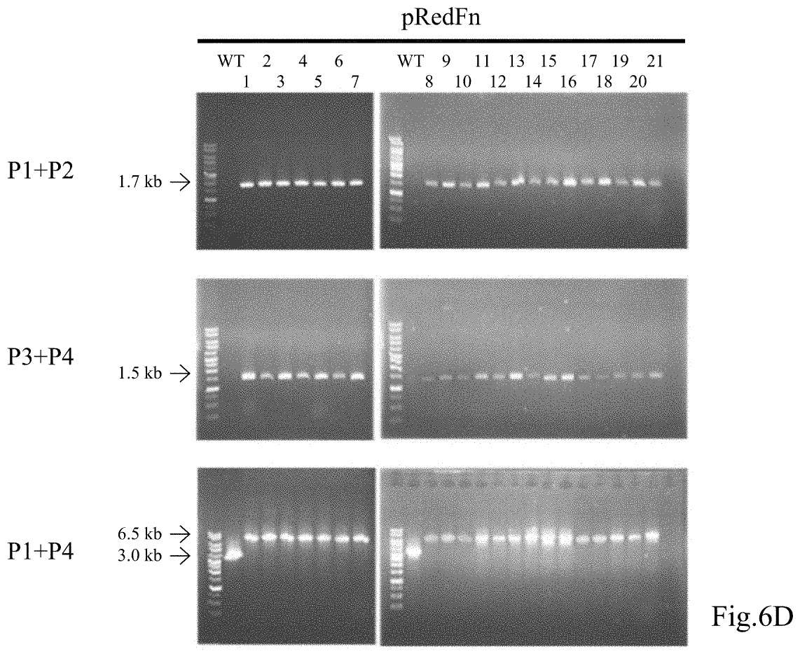

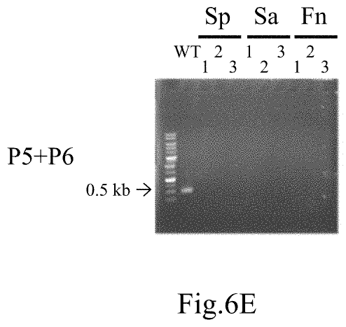

Further, a colony PCR is used to confirm whether the exogenous gene is integrated into a precise site in the chromosome of the Pseudomonas putida. Please refer to FIG. 6A, which is a schematic diagram showing the sites of 6 pairs of primers designed for the colony PCR. The primers of the colony PCR are designed at the junction of the chromosomes of Pseudomonas putida and the ends of the exogenous gene. The nucleotide sequence of primer P1 is referenced as SEQ ID NO: 23, the nucleotide sequence of primer P2 is referenced as SEQ ID NO: 24, the nucleotide sequence of primer P3 is referenced as SEQ ID NO: 25, the nucleotide sequence of primer P4 is referenced as SEQ ID NO: 26, the nucleotide sequence of primer P5 is referenced as SEQ ID NO: 27, and the nucleotide sequence of primer P6 is referenced as SEQ ID NO: 28. A size of left amplicon (P1+P2) is about 1.7 kb and the size of right amplicon (P3+P4) is about 1.5 kb. An amplicon of about 6.5 kb can be obtained by performing colony PCR with primers P1 and P4 PCR. In addition, the primers P5 and P6 are designed according to the original sequence on the chromosome of Pseudomonas putida. An amplicon of about 0.5 kb can be obtained by performing colony PCR with primers P5 and P6, which means that the exogenous gene has not been successfully integrated into the chromosome of Pseudomonas putida.

Please refer to FIGS. 6B, 6C and 6D, which show analytical results of the colony PCR for confirming the integration of the exogenous gene in the chromosome of the Pseudomonas putida. In FIGS. 6B, 6C and 6D, 21 colonies are selected in Example 1, Example 2 and Example 3, respectively, and colony PCR is performed with primers P1 and P2 or P3 and P4. The results show that although Pseudomonas putida has polyploid chromosomes, the exogenous gene of each of the selected colonies in Example 1, Example 2 and Example 3, which are edited with the gene editing system of Pseudomonas putida of the present disclosure, is successfully integrated in the target site. Thus, the gene editing system of Pseudomonas putida and the gene editing method thereof of the present disclosure can successfully edit the polyploid chromosomes of the Pseudomonas putida at the same time.

Please refer to FIG. 6E, which shows analytical result of the colony PCR for confirming the existence of the original chromosome sequence of the Pseudomonas putida after treating by the gene editing system of Pseudomonas putida. In FIG. 6E, 2 colonies are selected in Example 1, Example 2 and Example 3, respectively, and colony PCR is performed with primers P5 and P6. If the amplicon of about 0.5 kb is detected, it means that the Upp site of Pseudomonas putida that should have been replaced still exists. It also means that the exogenous gene is integrated in the wrong site or the exogenous gene is not completely integrated in all polyploid chromosomes of Pseudomonas putida. In FIG. 6E, no amplicon is detected in each of the selected colonies in Example 1, Example 2 and Example 3. The results indicate that the gene editing system of Pseudomonas putida and the gene editing method thereof of the present disclosure can successfully integrate the exogenous gene in the chromosome of Pseudomonas putida and can effectively replace the polyploid chromosomes of Pseudomonas putida.

### 4. The Gene Editing System of Pseudomonas putida Promotes the Homologous Recombination Efficiency in the Pseudomonas putida

To confirm the stability of the exogenous gene of the homologous recombinant obtained by the gene editing system of Pseudomonas putida and the gene editing method thereof of the present disclosure, the second transformant with the exogenous gene integrated in the correct site is cultured for another 6 days (after 144 generations), and the colony PCR is used to further verify whether the integrated exogenous gene still exists at the target site on the chromosome of Pseudomonas putida. In addition, quantitative analysis of the expression of the exogenous gene is performed by quantitative PCR.

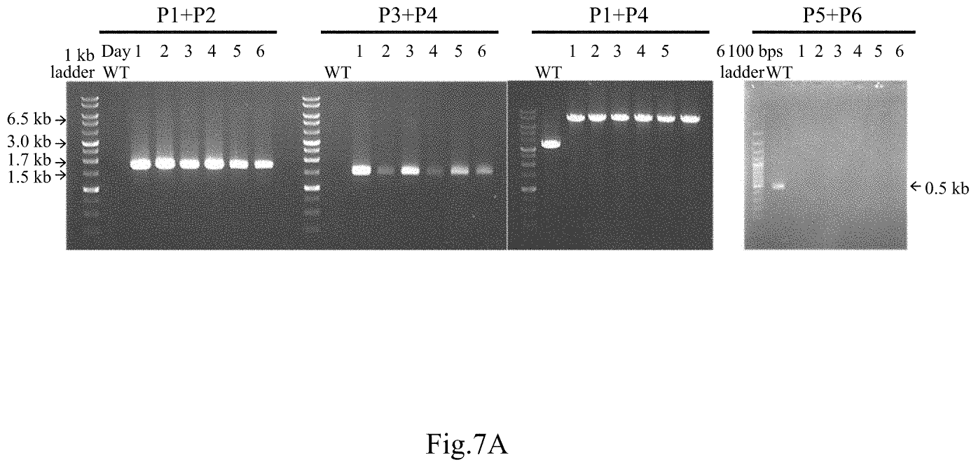

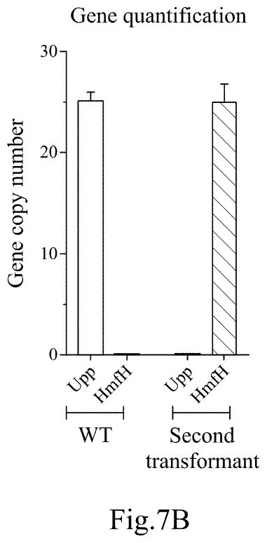

Please refer to FIGS. 7A and 7B. FIG. 7A shows analytical results of the colony PCR for confirming integration of the exogenous gene in the chromosome of the Pseudomonas putida after 6 days of treatment by the gene editing system of Pseudomonas putida. FIG. 7B is an analytical result showing gene copy number of the chromosome of the Pseudomonas putida which is integrated the exogenous gene.

In FIG. 7A, the exogenous gene is still present at the target site of the chromosome of each of the selected colonies in Example 1, Example 2 and Example 3 after 144 generations of cultivation. In FIG. 7B, after 144 generations of cultivation, the second transformant treated with the gene editing system of Pseudomonas putida of the present disclosure can stably express the exogenous gene (HmfH gene) in the 25 copies of chromosomes in Pseudomonas putida S12, and does not express the Upp gene of the original sequence of Pseudomonas putida. The results indicate that the strain edited by the gene editing system of Pseudomonas putida of the present disclosure can perform long-term stable expression of the integrated exogenous gene in the polyploid chromosomes.

Therefore, the gene editing system of Pseudomonas putida and the gene editing method thereof of the present disclosure, which incorporate the λ-Red system and the CRISPR system, can simultaneously and effectively trigger the double strand break at the target site of the genome on the polyploid chromosomes of Pseudomonas putida to cause the death of Pseudomonas putida, and can precisely integrate the exogenous gene with the genome of Pseudomonas putida by the exogenous gene expression plasmid. In addition, the exogenous gene can still be detected at the target site of Pseudomonas putida on the 6th day after the transformation, indicating that the homologous recombinant obtained by the gene editing system of Pseudomonas putida and the gene editing method thereof of the present disclosure can stably express the exogenous gene for a long time. In the conventional technology, the λ-Red system is mostly used in Escherichia coli and is not used in Pseudomonas putida for gene editing. Therefore, the gene editing system of Pseudomonas putida and the gene editing method thereof of the present disclosure are novel and effective for gene editing of Pseudomonas putida, which can break through the limitations of the conventional technology and can shorten gene editing time and freely select the target site on the chromosome of Pseudomonas putida that needs to be modified, while promoting the efficiency of gene editing of Pseudomonas putida.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A gene editing system of Pseudomonas putida, comprising:
a Pseudomonas putida;
a RedCas expression plasmid, which successively comprises a first replication origin, a first antibiotic resistance gene, a λ-Red expression cassette and a Cas expression cassette, wherein the first replication origin comprises a nucleic acid sequence of SEQ ID NO: 1, the λ-Red expression cassette comprises a first promoter, a Gam gene, a Bet gene and an Exo gene, and the Cas expression cassette comprises a second promoter and a Cas gene; and
an exogenous gene expression plasmid, which successively comprises a second replication origin, a left homology arm, a second antibiotic resistance gene, an exogenous gene expression cassette, a right homology arm and a gRNA cassette, wherein the exogenous gene expression cassette comprises a third promoter and an exogenous gene, the gRNA cassette comprises a fourth promoter and a gRNA sequence, and the gRNA sequence is composed of a spacer and a scaffold;
wherein the left homology arm and the right homology arm compose a homology region, a sequence of the homology region is homologous to a first specific sequence of a chromosome of the Pseudomonas putida, a sequence of the spacer is homologous to a second specific sequence of the chromosome of the Pseudomonas putida, and the first antibiotic resistance gene and the second antibiotic resistance gene are different.

2. The gene editing system of Pseudomonas putida of claim 1, wherein the RedCas expression plasmid further comprises an araC gene, and the first promoter is an araBAD promoter.

3. The gene editing system of Pseudomonas putida of claim 1, wherein the Cas gene comprises a nucleic acid sequence of SEQ ID NO: 9, a nucleic acid sequence of SEQ ID NO: 10 or a nucleic acid sequence of SEQ ID NO: 11.

4. The gene editing system of Pseudomonas putida of claim 1, wherein the exogenous gene expression plasmid further comprises two Flp/FRT knockout sequences, and the second antibiotic resistance gene is located between the two Flp/FRT knockout sequences.

5. A gene editing method of Pseudomonas putida, comprising:
constructing a RedCas expression plasmid, which successively comprises a first replication origin, a first antibiotic resistance gene, a λ-Red expression cassette and a Cas expression cassette, wherein the first replication origin comprises a nucleic acid sequence of SEQ ID NO: 1, the λ-Red expression cassette comprises a first promoter, a Gam gene, a Bet gene and an Exo gene, and the Cas expression cassette comprises a second promoter and a Cas gene;
constructing an exogenous gene expression plasmid, which successively comprises a second replication origin, a left homology arm, a second antibiotic resistance gene, an exogenous gene expression cassette, a right homology arm and a gRNA cassette, wherein the exogenous gene expression cassette comprises a third promoter and an exogenous gene, the gRNA cassette comprises a fourth promoter and a gRNA sequence composed of a spacer and a scaffold, the left homology arm and the right homology arm compose a homology region, a sequence of the homology region is homologous to a first specific sequence of a chromosome of a Pseudomonas putida, a sequence of the spacer is homologous to a second specific sequence of the chromosome of the Pseudomonas putida, and the first antibiotic resistance gene and the second antibiotic resistance gene are different;
transforming the RedCas expression plasmid into the Pseudomonas putida to obtain a first transformant;
performing an induction step, wherein the first transformant is cultured and then an inducer is added to induce the RedCas expression plasmid expresses a Gam protein, a Beta protein, an Exo protein and a Cas protein to obtain an induced first transformant; and
transforming the exogenous gene expression plasmid into the induced first transformant to obtain a second transformant, so that the exogenous gene expression plasmid expresses a gRNA, wherein the gRNA and the Cas protein form a Cas protein complex triggering a double strand break on the second specific sequence of the chromosome of the second transformant, and the Gam protein, the Beta protein and the Exo protein co-guide the homology region of the exogenous gene expression plasmid to perform homologous recombination with the first specific sequence of the chromosome of the second transformant, so that the exogenous gene is integrated into the first specific sequence of the chromosome of the second transformant.

6. The gene editing method of Pseudomonas putida of claim 5, wherein in the induction step, the inducer is added after the first transformed strain is cultivated to a stationary phase.

7. The gene editing method of Pseudomonas putida of claim 5, wherein the RedCas expression plasmid further comprises an araC gene, and the first promoter is an araBAD promoter.

8. The gene editing method of Pseudomonas putida of claim 7, wherein the inducer is arabinose.

9. The gene editing method of Pseudomonas putida of claim 5, further comprising a first selection step, wherein the first transformant is cultured in a first medium containing a first antibiotic.

10. The gene editing method of Pseudomonas putida of claim 5, further comprising a second selection step, wherein the second transformant is cultured in a second medium containing a second antibiotic.
